(19) United States
(12) Patent Application Publication
(10) Pub. No.: US 2014/0378333 A1
(43) Pub. Date: Dec. 25, 2014

(54) DIGITAL BRIDGE PCR

(71) Applicants: David R. Walt, Boston, MA (US); David C. Duffy, Somerville, MA (US)

(72) Inventors: David R. Walt, Boston, MA (US); David C. Duffy, Somerville, MA (US)

(73) Assignees: Tufts University, Medford, MA (US); Quantarex Corporation, Lexington, MA (US)

(21) Appl. No.: 14/344,739

(22) PCT Filed: Sep. 13, 2012
(86) PCT No.: PCT/US12/55174

§ 371 Date: May 30, 2014

Related U.S. Application Data
(60) Provisional application No. 61/534,358, filed on Sep. 13, 2011.

(51) Int. Cl. C12Q 1/68

(52) U.S. Cl. CPC C12Q 1/686; C12Q 1/6876; C12Q 2600/16
USPC 506/9; 506/16

(57) ABSTRACT

The present disclosure, among other things, provides methodologies for quantifying targets of interest in samples by 1) capturing single target entities on individual solid phase particles in a manner that permits that those particles that contain captured targets to be optically distinguished from those that do not, and 2) optically analyzing the particles so that those with captured target entities are “counted”. In some embodiments, provided methods and compositions in the present application comprise a population of particles including one or more sub-populations distinguishable from one another.

### RELATED REFERENCES

This application claims priority to U.S. provisional patent application Ser. No. 61/534,358, filed Sep. 13, 2011, the entire contents of which are herein incorporated by reference.

### BACKGROUND

Detection and quantification of nucleic acids is of fundamental importance in the fields of genetic and medical research, clinical chemistry, and forensic science, among others. Significant effort is invested in the development of new techniques, with a particular focus on improving sensitivity of detection and/or precision of quantification.

### SUMMARY

The present invention provides methodologies for quantifying targets of interest in samples by 1) capturing single target entities on individual solid phase particles in a manner that permits that those particles that contain captured targets to be optically distinguished from those that do not, and 2) optically analyzing the particles so that those with captured target entities are “counted”. In many embodiments, the present invention provides methodologies that include amplification on the particles of captured target entities, and/or of an optical signal or characteristic associated or correlated with their capture.

The present invention, among other things, provides the insight that bridge polymerase chain reaction (PCR) technologies can beneficially be employed to quantify target nucleic acids present in samples.

Separately and additionally, the present invention provides the insight that bridge PCR technologies are sufficiently robust to permit rapid and/or precise quantification even of relatively crude samples, with minimal nucleic acid processing steps.

Still further, the present invention also provides the insight that use of bridge PCR technologies with optically encoded particle technologies permits simultaneous quantification of a plurality of different nucleic acids within a sample through optical analysis of particle populations.

In some embodiments, provided methodologies enable quantification of nucleic acids present in a sample at a single-molecule level or at a level of subfemtomolar concentration. The present invention encompasses the recognition that one challenge with many commonly employed single molecule analysis methodologies is that the extreme dilutions utilized to ensure that only a single molecule is present can make it difficult to isolate enough molecules for detection, as the probability of finding a single molecule (e.g., a target nucleic acid) in a dilute solution is low. The present invention not only identifies the source of this problem, but provides a solution by providing technologies that capture individual nucleic acid molecules that are present in dilute solution onto separate solid phases, which can then be optionally concentrated. The present invention further provides for amplification of captured nucleic acids, and/or of an optical signal or characteristic associated or correlated with them, and/or for optical detection of nucleic-acid-associated solid phase (and/or of amplified nucleic acids).

### BRIEF DESCRIPTION OF THE DRAWING

FIG. 1 shows a schematic of a typical Bridge PCR.

FIG. 2 illustrates an exemplary particle with a captured target nucleic acid according to the present invention.

### DEFINITIONS

In order for the present disclosure to be more readily understood, certain terms are first defined below. Additional definitions for the following terms and other terms are set forth throughout the specification.

In this application, the use of “or” means “and/or” unless stated otherwise. As used in this application, the term “comprise” and variations of the term, such as “comprising” and “comprises,” are not intended to exclude other additives, components, integers or steps. As used in this application, the terms “about” and “approximately” are used as equivalents. Any numerals used in this application with or without about/approximately are meant to cover any normal fluctuations appreciated by one of ordinary skill in the relevant art. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

“Associated”: As used herein, the term “associated” typically refers to two or more entities in physical proximity with one another, either directly or indirectly (e.g., via one or more additional entities that serve as a linking agent), to form a structure that is sufficiently stable so that the entities remain in physical proximity under relevant conditions, e.g., physiological conditions. In some embodiments, associated moieties are covalently linked to one another. In some embodiments, associated entities are non-covalently linked. In some embodiments, associated entities are linked to one another by specific non-covalent interactions (i.e., by interactions between interacting ligands that discriminate between their interaction partner and other entities present in the context of use, such as, for example. streptavidin/avidin interactions, antibody/antigen interactions, etc.). Alternatively or additionally, a sufficient number of weaker non-covalent interactions can provide sufficient stability for moieties to remain associated. Exemplary non-covalent interactions include, but are not limited to, affinity interactions, metal coordination, physical adsorption, host-guest interactions, hydrophobic interactions, pi stacking interactions, hydrogen bonding interactions, van der Waals interactions, magnetic interactions, electrostatic interactions, dipole-dipole interactions, etc.

“Beads”, “microspheres”, or “particles”: The terms “beads”, “microspheres”, or “particles” as used herein, refer to discrete solid phases, and may be used interchangeably. Such solid phases can be of any shape or size. Any of a variety of materials can be used to form or provide particles, as will be understood by those of skill in the art. In some embodiments, particular materials and/or shapes may be preferred based on chemistries or other features utilized in the relevant embodiments; those of ordinary skill will be well familiar with options and parameters guiding selection. In many embodiments, suitable materials include but not limited to, plastics, ceramics, glass, polystyrene, methylstyrene, acrylic polymers, metal, paramagnetic materials, thoria sol, graphitic carbon, titanium dioxide, latex or cross-linked dextrans such as Sepharose, cellulose, nylon, cross-linked micelles and teflon.

“Nucleic acid”: The term “nucleic acid” as used herein, refers to a polymer of nucleotides. In some embodiments, nucleic acids are or contain deoxyribonucleic acids (DNA); in some embodiments, nucleic acids are or contain ribonucleic acids (RNA). In some embodiments, nucleic acids include naturally-occurring nucleotides (e.g., adenosine, thymidine, guanosine, cytidine, uridine, deoxyadenosine, deoxythymidine, deoxyguanosine, and deoxycytidine). Alternatively or additionally, in some embodiments, nucleic acids include non-naturally-occurring nucleotides including, but not limited to, nucleoside analogs (e.g., 2-aminoadenosine, 2-thiothymidine, inosine, pyrrolo-pyrimidine, 3-methyl adenosine, C5-propynylcytidine, C5-propynyluridine, C5-bromouridine, C5-fluorouridine, C5-iodouridine, C5-methylcytidine, 7-deazaadenosine, 7-deazaguanosine, 8-oxoadenosine, 8-oxoguanosine, O(6)-methylguanine, and 2-thiocytidine), chemically modified bases, biologically modified bases (e.g., methylated bases), intercalated bases, modified sugars (e.g., 2′-fluororibose, ribose, 2′-deoxyribose, arabinose, and hexose), or modified phosphate groups. In some embodiments, nucleic acids include phosphodiester backbone linkages; alternatively or additionally, in some embodiments, nucleic acids include one or more non-phosphodiester backbone linkages such as, for example, phosphorothioates and 5′-N-phosphoramidite linkages. In some embodiments, a nucleic acid is an oligonucleotide in that it is relatively short (e.g., less that about 5000, 4000, 3000, 2000, 1000, 900, 800, 700, 600, 500, 450, 400, 350, 300, 250, 200, 150, 100, 90, 80, 70, 60, 50, 45, 40, 35, 30, 25, 20, 15, 10 or fewer nucleotides in length).

“A target nucleic acid”, or “target nucleic acids”: The terms “a target nucleic acid” or “target nucleic acids” as used herein, refer to one or more nucleic acid molecules to be detected and/or quantified in accordance with the present invention. Exemplary target nucleic acids include, but not limited to DNA, RNA, miRNA, and cDNAs. In some embodiments, a target nucleic acid comprises a plurality of different nucleic acid molecules (i.e., having different nucleotide sequences); in some embodiments, only a single nucleic acid is a target. In some embodiments, target nucleic acids are of the same origin (e.g., from the same chromosome, genomic locus, or gene, although the molecules may come from one individual, or multiple individuals, or more than one type of cells, such as tumor cells, placental cells, blood cells, etc.).

“Sample”: The term “sample” refers to a volume or mass obtained, provided, and/or subjected to analysis. In some embodiments, a sample is or comprises a tissue sample, cell sample, a fluid sample, and the like. In some embodiments, a sample is taken from a subject (e.g., a human or animal subject). In some embodiments, a tissue sample is or comprises brain, hair (including roots), buccal swabs, blood, saliva, semen, muscle, or from any internal organs, or cancer, precancerous, or tumor cells associated with any one of these. A fluid may be, but is not limited to, urine, blood, ascites, pleural fluid, spinal fluid, and the like. A body tissue can include, but is not limited to, brain, skin, muscle, endometrial, uterine, and cervical tissue or cancer, precancerous, or tumor cells associated with any one of these. In an embodiment, a body tissue is brain tissue or a brain tumor or cancer. Those of ordinary skill in the art will appreciate that, in some embodiments, a “sample” is a “primary sample” in that it is obtained from a source (e.g., a subject); in some embodiments, a “sample” is the result of processing of a primary sample, for example to remove certain potentially contaminating components and/or to isolate or purify certain components of interest.

“Substantially”: As used herein, the term “substantially”, and grammatic equivalents, refer to the qualitative condition of exhibiting total or near-total extent or degree of a characteristic or property of interest. One of ordinary skill in the art will understand that biological and chemical phenomena rarely, if ever, go to completion and/or proceed to completeness or achieve or avoid an absolute result.

### DETAILED DESCRIPTION OF CERTAIN EMBODIMENTS

### Digital PCR

The term “digital” is used in the art of nucleic acid analysis to refer to the detection and/or processing of single nucleic acid molecules. Digital polymerase chain reaction (“digital PCR”), thus involves amplification (i.e., copying) of individual isolated template molecules.

According to common methodologies, digital PCR is carried out by first separating template molecules from one another and isolating them in distinct reaction areas (e.g., wells, chambers, droplets, etc). Typically, a sample containing potential template nucleic acid molecules (i.e., nucleic acid molecules to be amplified) is provided, and then is diluted dramatically and separated into a set of reaction areas so that each reaction area contains, on average, only one or zero template molecules. PCR amplification is performed on the reaction areas, and templates are amplified if they are present.

Those of ordinary skill in the art will be aware of a wide range of established formats for performing PCR in general, and digital PCR in particular. For example, methodologies are available for performing PCR in solution, on solid phases, in emulsions, etc..

The present invention provides a novel format for digital PCR, and in particular utilizes so-called “bridge PCR” for digital analysis. As will be familiar to those of skill in the art, the term “bridge PCR” is a technology that uses primer pairs (or sets) bound to a solid phase for the extension and amplification of solution phase target nucleic acid molecules. The name refers to the fact that during an annealing step, the extension product from one primer forms a bridge to the other primer. Amplified products are bound to the surface of the solid phase.

As a person of ordinary skill in the art will appreciate, bridge PCR reactions typically utilize primer pairs/sets that are separated from one another on the solid phase by a distance that is less than the length of a target nucleic acid to be amplified. One primer of a pair (e.g., the “first” primer of a pair or the capture primer) hybridizes with target in solution, and “captures” it to the particle. That primer is then extended to generate a complementary strand, which hybridizes with the other primer (e.g., the “second” primer) of the pair. The amplified (and double stranded) product is therefore “attached” to the particle by covalent linkage of its strands with the attached primers (and by hybridization with complementary primers).

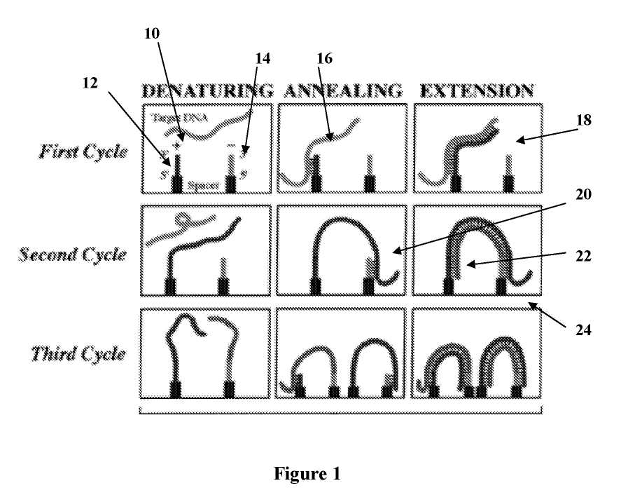

FIG. 1 presents an illustration of a typical bridge PCR reaction on a solid phase. As can be seen with reference to FIG. 1, at least one primer pair (typically covalently linked to a solid phase) is contacted with a sample that may contain a target nucleic acid. The target nucleic acid (10) hybridizes with a first primer (12) of the primer pair to form a hybridization product (16). Those of ordinary skill in the art will appreciate that the target nucleic acid (10) may initially be single or double stranded. In principle, an individual double-stranded target molecule could be captured for bridge PCR by either primer of a primer pair; in some embodiments, each strand may be separately captured. In many bridge PCR methodologies, target nucleic acids are rendered single-stranded prior to or during contact with primer pairs. Regardless of whether only a single strand is captured, which strand is captured, or both strands are captured, the primer(s) with which the target nucleic acid hybridizes is/are referred to herein as the “capture primer(s)” (e.g., 12 in FIG. 1).

Once a target nucleic acid/strand (10) is captured, a capture primer is extended so that a strand complementary to the captured stand is produced. The captured strand and the complementary strand (18) can be denatured from one another. This complementary strand can then hybridize with a second primer (14) of the relevant primer pair, so that a bridged hybridization product (20) is generated. The second primer (14) can be extended so that a strand complementary to the complementary strand (18) is produced. The new complementary strand (22) and the complementary strand (18) generate a bridged amplification product (24).

The new complementary strand (22) and the complementary strand (18) can then be denatured from one another for subsequent rounds of primer extension/hybridization, resulting in amplification of the target nucleic acid (10) on the particle. For further details of conventional bridge PCR technologies, see, for example, U.S. Pat. No. 5,641,658, “Method For Performing Amplification of Nucleic Acid with Two Primer Bound To a Single Solid Support”, and Bing, D. H., et al., Bridge Amplification: “A Solid Phase PCR System for the Amplification and Detection of Allelic Differences in Single Copy Genes”, Proceedings of the Seventh International Symposium on Human Identification, Promega Corporation 1996-1998, which are both incorporated by reference.

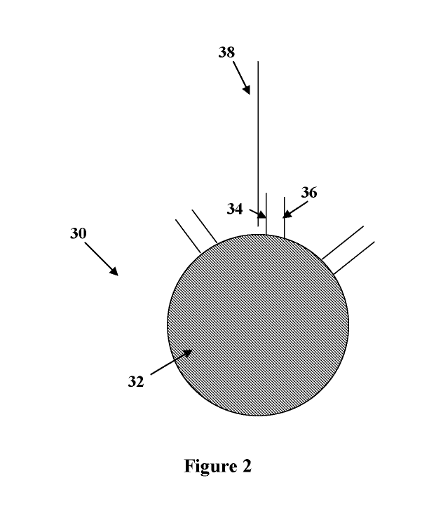

As described herein, one feature of the present invention is the recognition that bridge PCR technologies can be usefully utilized in digital applications. That is, according to the present invention, a contacting step, for bridge amplification and optionally following developments, is performed so that, on average, not more than one copy of a target nucleic acid (38) is captured by a capture primer (34) to an individual particle (32) as illustrated in FIG. 2.

In some embodiments, an individual particle contains only one or more copies of a single type of primer pair/set, designed or selected to capture and/or amplify only a single type of target nucleic acid.

In some embodiments, an individual particle may contain a plurality of primer pairs/sets (e.g., one or more copies of different types of primer pairs/sets) so that more than one different type of target nucleic acids (i.e., target nucleic acids of different sequences) can be captured and/or amplify on the same particle; in such embodiments, a contacting step is performed so that, on average, not more than one copy of each target molecule hybridizes with an individual particle. In some embodiments, where different types of primer pairs/sets are employed, some or all of the different types of target nucleic acids have related sequences (e.g., sequences that share a specified degree of sequence identity and/or at least one common sequence element, but that differ in length and/or, to some degree, sequence). In some embodiments, some or all of the different types of target nucleic acids have sequences that are unrelated to one another. In some embodiments, some or all of the different types of target nucleic acids share a common biological feature (e.g., are found in infectious agents such as viruses, microbes, etc).

In some embodiments, no free primers (e.g., primers in solution and not immobilized on a particle) are used. Without being bound to any particular theory, Applicant notes that use of any immobilized primers, and no free primers in solution, can restrict PCR amplification so that it is restricted to the particles.

In many embodiments, where it is desirable to simultaneously detect and/or quantify a plurality of different types of target nucleic acids, such simultaneous detection and/or quantification is achieved by utilizing a plurality of different particle subpopulations (as discussed more fully below), where each subpopulation contains individual particles that have primer pairs/sets designed and/or selected to capture and/or amplify only a single type of target nucleic acid, rather than by employing individual particles that contain pluralities of primer pairs/sets. In many such embodiments, notwithstanding that multiple primer pairs/sets are employed, utilized primers may have nucleotide sequences selected to hybridize specifically with predetermined target nucleic acids of known nucleotide sequence.

Those of ordinary skill in the art will appreciate that any of a variety of techniques can be utilized to ensure that, on average, not more than one copy of a target nucleic acid (or, on average, not more than one copy of a target nucleic acid of each type that can be captured by an individual particle) hybridizes to any individual particle. In many embodiments, sample is diluted to achieve this result. In some embodiments, however, one or more concentration steps may be involved. Alternatively or additionally, steps may be taken to remove one or more non-target nucleic acids (e.g., particularly abundant nucleic acid molecules, nucleic acid molecules of a particular type [e.g., DNA vs RNA], nucleic acid molecules within a particular size range, and/or nucleic acid molecules that might be expected to interfere with proper or ready capture of target nucleic acid molecules) from a sample prior to or during the contacting step. Alternatively or additionally, one or more target or non-target nucleic acids may be added to a sample prior to or during the contacting step. In some such embodiments, a detectable control nucleic acid is added to the sample.

In some embodiments, once target nucleic acid(s) is/are hybridized to particles, sample is removed (e.g., by washing) and/or particles are concentrated.

According to the present invention, in many embodiments, amplification comprises performing one or more rounds of bridge PCR on the particles. In some embodiments, about 1, about 5, about 10, about 20, about 25, about 30, about 35, about 40, about 45, about 50, about 100 rounds or more of bridge PCR extension are performed. Without limiting the present invention to any particular rounds of amplification in bridge PCR, in some embodiments, amplification can be performed until a property of a particle is changed and detectable, so that, for example, such particles can be distinguished from those that do not contain captured and/or amplified target nucleic acids.

### Samples

In general, any sample containing at least one target nucleic acid whose presence is to be detected and/or whose amount is to be quantified, can be utilized in accordance with the present invention. In many embodiments, a sample contains an unknown amount of target nucleic acid(s), and may not contain any target nucleic acid.

In some embodiments, a sample is crude and/or unprocessed. Crude samples can include, for example, cells, blood, saliva, urine, feces, anorectal swabs, vaginal swabs, cervical swabs, and the like. Unprocessed samples, in some embodiments, can be any samples without any purification/isolation processes. In some embodiments, a sample is considered to be “crude” if it is a primary sample from a source, or contains target nucleic acid at a concentration that is not more than 2, 3, 4, or 5-fold concentrated as compared with a primary sample from the source. The ability to use crude/unprocessed samples in certain embodiments of the present invention may eliminate the need for extensive, expensive, and possible contaminating procedures (e.g., DNA extraction) and allow for faster turnaround times from sampling to results.

In some embodiments, a sample is from a natural source (e.g., environmental sample, biological sample, etc.). In some embodiments, a sample is a clinical or forensic sample. In some embodiments, a sample may contain live cells, or cells that are non-replicating or dead or in a vegetative state (for example, vegetative bacteria or spores). In some embodiments, a sample may contain viruses.

A sample can be pre-treated. In some embodiments, a sample contains purified target nucleic acids. In some embodiments, a sample is spiked with reference nucleic acids. In some embodiments, a sample is spiked with a detectable reference, for example, present at a known amount.

In some embodiments, the methods described herein are used to detect and/or quantify the very small amount of a target nucleic acid in a sample. For example, the amount of a target nucleic acid can be less than 1 pg, 50 pg, 500 pg, 1 ng, 50 ng, 500 ng, 1 ug, 50 ug, 500 ug or 1 mg.

### Target Nucleic Acids

In general, target nucleic acids may be any form of DNA, RNA, or any combination thereof. In certain embodiments of the present invention, a target nucleic acid may be or contain a portion of a gene, a regulatory sequence, genomic DNA, cDNA, RNA including mRNA and rRNA, or any combination thereof. In some embodiments, a target nucleic acid may be or contain a single or double stranded RNA or DNA, including, for example, gDNA, cDNA, mRNA, pre-mRNA, miRNA, etc. Furthermore, in some embodiments, a target nucleic acid may include one or more residues that is an analog of a naturally-occurring residue. In some embodiments, such analogs have a backbone other than a phosphodiester backbone. For example, the so-called “peptide nucleic acids,” which are known in the art and have peptide bonds instead of phosphodiester bonds in the backbone, may be considered to be “target nucleic acids” in accordance with certain embodiments of the invention.

Target nucleic acids can be naturally or synthetically produced, including produced using recombinant expression systems, chemically synthesized, etc. Where appropriate, e.g., in the case of chemically synthesized molecules, nucleic acids can comprise nucleoside analogs such as analogs having chemically modified bases or sugars, backbone modifications, etc. In some embodiments, a nucleic acid is or comprises natural nucleosides (e.g. adenosine, thymidine, guanosine, cytidine, uridine, deoxyadenosine, deoxythymidine, deoxyguanosine, deoxycytidine and hydroxymethylcytosine); nucleoside analogs (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, O(6)-methylguanine, and 2-thiocytidine); chemically modified bases; biologically modified bases (e.g., methylated bases); intercalated bases; modified sugars (e.g., 2′-fluororibose, ribose, 2′-deoxyribose, arabinose, and hexose); and/or modified phosphate groups (e.g., phosphorothioates and 5′-N-phosphoramidite linkages).

In some embodiments, the present invention may be directed to “unmodified nucleic acids,” meaning nucleic acids (e.g. polynucleotides and residues, including nucleotides and/or nucleosides) that have not been chemically modified in a crude and unprocessed sample. In some embodiments, targeted nucleic acids can be truncated. For example, targeted nucleic acids can be truncated versions of their natural state, or targeted nucleic acids can be truncated from their initially produced forms but not otherwise structurally altered.

A target nucleic acid, in various embodiments, can be one that is found in a biological organism (including, for example, a microorganism or infectious agent, or any naturally occurring, bioengineered or synthesized component thereof.

In accordance with the present invention, in many embodiments, the target nucleic acid is particularly identified and/or known in advance, and primer sets are designed and/or selected to specifically interact with the identified target nucleic acid, and not with other nucleic acids that may be in the sample. Thus, in many embodiments, one or both primers in primer sets have nucleotide sequences selected to hybridize specifically with a predetermined target nucleic acid of known nucleotide sequence.

miRNAs

In some embodiments, provided methods herein are used to detect and/or quantify miRNAs. miRNAs can be found in genomes of humans, animals, plants and viruses. According to the present invention, a target nucleic acid, in some embodiments, can be or comprise one or more miRNAs that is/are generated from endogenous hairpin-shaped transcripts. In some embodiments, a target nucleic acid can be or comprise one or more miRNAs that is/are transcribed as long primary transcripts (pri-microRNAs), for example, by RNA polymerase II enzyme in animals. There are a total of 475 human miRNA genes currently listed in the miRNA database (http://microrna.sanger.ac.uk/sequences/ftp.shtml) and there are predictions that this number will go up to approximately 1000, which would be equivalent to almost 3% of protein-coding genes. Many miRNAs are thought to be important in the regulation of gene expression.

### Particles

Where particles are used in the practice of the present invention, it is not intended that the present invention be limited to a particular type. A variety of particle types are commercially available, including but not limited to, particles selected from agarose beads, streptavidin-coated beads, NeutrAvidin-coated beads, antibody-coated beads, paramagnetic beads, magnetic beads, electrostatic beads, electrically conducting beads, fluorescently labeled beads, colloidal beads, glass beads, semiconductor beads, and polymeric beads.

Particles useful in accordance with the present invention need not be spherical; irregular particles and/or particles having non-spherical shapes, may be used.

In general, a particle in accordance with the present invention is typically an entity having a greatest dimension (e.g. diameter) of less than 1000 microns (μm). In some embodiments, particles have a greatest dimension of less than 500 μm, 200 μm, 100 μm, 50 μm, 10 μm, 5 μm or 1 μm. In some embodiments, particles have a greatest dimension of less than 900 nm, 800 nm, 700 nm, 600 nm, 500 nm, 400 nm, 300 nm, 200 nm, or 100 nm. Smaller particles, e.g., having a greatest dimension of 50 nm or less are used in some embodiments of the invention. In some embodiments, particles have a greatest dimension ranging between 1 μm and 10 μm. In some embodiments, particles have a greatest dimension ranging between any two values above.

A population of particles can be but need not be relatively uniform in terms of size, shape, and/or composition. Particles can have a variety of different shapes including spheres, oblate spheroids, cylinders, ovals, ellipses, shells, cubes, cuboids, cones, pyramids, rods (e.g., cylinders or elongated structures having a square or rectangular cross-section), tetrapods (particles having four leg-like appendages), triangles, prisms, etc.

Particles can be solid or hollow and can comprise one or more layers (e.g., nanoshells, nanorings, etc.). Particles may have a core/shell structure, wherein the core(s) and shell(s) can be made of different materials. Particles may comprise gradient or homogeneous alloys. Particles may be composite particles made of two or more materials, of which one, more than one, or all of the materials possess magnetic properties, electrically detectable properties, and/or optically detectable properties.

In certain embodiments of the invention, a particle is porous, by which is meant that the particle contains holes or channels, which are typically small compared with the size of a particle. For example a particle may be a porous silica particle, e.g., a mesoporous silica particle or may have a coating of mesoporous silica.

In some embodiments, particles are biocompatible. Additionally or alternatively, particles may have a coating layer. Use of a biocompatible coating layer can be advantageous in some embodiments. Suitable coating materials include, but are not limited to, natural proteins such as bovine serum albumin (BSA), biocompatible hydrophilic polymers such as polyethylene glycol (PEG) or a PEG derivative, phospholipid-(PEG), silica, lipids, polymers, carbohydrates such as dextran, other materials that can be associated with particles, etc. Coatings may be applied or assembled in a variety of ways such as by dipping, using a layer-by-layer technique, by self-assembly, conjugation, etc.

In some embodiments, polymeric particles may be used in accordance with the present invention. For example, particles can be made of organic polymer including, but not limiting to, polystyrene, polymethylmethacrylate, polyacrylamide, poly(vinyl chloride), carboxylated poly(vinyl chloride), poly(vinyl chloride-co-vinyl acetate-co-vinyl alcohol), and combination thereof. Additionally or alternatively, particles can be or comprises inorganic polymers such as silica (SiO₂).

Additionally or alternatively, particles can be functionalized (e.g., surface functionalized by adsorption or covalently bonding) or “doped” or “loaded” with fluorescent and luminescent moieties (e.g., fluorescent dyes) for optical encoding of particles. Examples of fluorescent dyes include fluorescein, rhodamine, acridine dyes, Alexa dyes, cyanine dyes, etc. Fluorescent and luminescent moieties may include a variety of naturally occurring proteins and derivatives thereof, e.g., genetically engineered variants. For example, fluorescent proteins include green fluorescent protein (GFP), enhanced GFP, red, blue, yellow, cyan, and sapphire fluorescent proteins, reef coral fluorescent protein, etc. Luminescent proteins include luciferase, aequorin and derivatives thereof. In addition to or alternative to single optical moieties, encoding can be accomplished in a ratio of at least two moieties.

In some embodiments, particles are or comprise intrinsically fluorescent or luminescent particles. In certain embodiments, particles are or comprise quantum dots (QDs). QDs are bright, fluorescent nanocrystals with physical dimensions small enough such that the effect of quantum confinement gives rise to unique optical and electronic properties. Semiconductor QDs are often composed of atoms from groups II-VI or III-V in the periodic table, but other compositions are possible. By varying their size and composition, the emission wavelength can be tuned (i.e., adjusted in a predictable and controllable manner) from the blue to the near infrared. QDs generally have a broad absorption spectrum and a narrow emission spectrum. Thus different QDs having distinguishable optical properties (e.g., peak emission wavelength) can be excited using a single source. In general, QDs are brighter and photostable than most conventional fluorescent dyes. QDs and methods for their synthesis are well known in the art (see, e.g., U.S. Pat. Nos. 6,322,901; 6,576,291; and 6,815,064; all of which are incorporated herein by reference). QDs can be rendered water soluble by applying coating layers comprising a variety of different materials (see, e.g., U.S. Pat. Nos. 6,423,551; 6,251,303; 6,319,426; 6,426,513; 6,444,143; and 6,649,138; all of which are incorporated herein by reference). For example, QDs can be solubilized using amphiphilic polymers. Exemplary polymers that have been employed include octylamine-modified low molecular weight polyacrylic acid, polyethylene-glycol (PEG)-derivatized phospholipids, polyanhydrides, block copolymers, etc.

Exemplary QDs suitable for use in accordance with the present invention in some embodiments, include ones with a wide variety of absorption and emission spectra and they are commercially available, e.g., from Quantum Dot Corp. (Hayward Calif.; now owned by Invitrogen) or from Evident Technologies (Troy, N.Y.). For example, QDs having peak emission wavelengths of approximately 525 nm, approximately 535 nm, approximately 545 nm, approximately 565 nm, approximately 585 nm, approximately 605 nm, approximately 655 nm, approximately 705 nm, and approximately 800 nm are available. Thus QDs can have a range of different colors across the visible portion of the spectrum and in some cases even beyond.

In certain embodiments, optically detectable particles are or comprise metal particles. Metals of use include, but are not limited to, gold, silver, iron, cobalt, zinc, cadmium, nickel, gadolinium, chromium, copper, manganese, palladium, tin, and alloys thereof. Oxides of any of these metals can be used.

In certain embodiments, optical detectable particles comprise a hologram.

Certain metal particles, referred to as plasmon resonant particles, exhibit the well known phenomenon of plasmon resonance. The features of the spectrum of a plasmon resonant particle (e.g., peak wavelength) depend on a number of factors, including the particle's material composition, the shape and size of the particle, the refractive index or dielectric properties of the surrounding medium, and the presence of other particles in the vicinity. Selection of particular particle shapes, sizes, and compositions makes it possible to produce particles with a wide range of distinguishable optically detectable properties thus allowing for concurrent detection of multiple nucleic acids by using particles with different properties such as peak scattering wavelength.

Magnetic properties of particles can be used in accordance with the present invention. Particles in some embodiments are or comprise magnetic particles, that is, magnetically responsive particles that contain one or more metals or oxides or hydroxides thereof. Magnetic particles may comprise one or more ferrimagnetic, ferromagnetic, paramagnetic, and/or superparamagnetic materials. Useful particles may be made entirely or in part of one or more materials selected from the group consisting of: iron, cobalt, nickel, niobium, magnetic iron oxides, hydroxides such as maghemite (γ-Fe₂O₃), magnetite (Fe₃O₄), feroxyhyte (FeO(OH)), double oxides or hydroxides of two- or three-valent iron with two- or three-valent other metal ions such as those from the first row of transition metals such as Co(II), Mn(II), Cu(II), Ni(II), Cr(III), Gd(III), Dy(III), Sm(III), mixtures of the afore-mentioned oxides or hydroxides, and mixtures of any of the foregoing. See, e.g., U.S. Pat. No. 5,916,539 (incorporated herein by reference) for suitable synthesis methods for certain of these particles. Additional materials that may be used in magnetic particles include yttrium, europium, and vanadium.

### Digital Detection

In many embodiments of the present invention, a single copy of a target nucleic acid is captured on a particle, and is amplified for example, by bridge PCR, so that one or more properties (or aspects) of the particle are changed, and particles containing captured nucleic acids are distinguishable from those not containing captured nucleic acids.

Any appropriate means and/or system can be utilized in accordance with the present invention to detect and/or quantify such captured-target-containing particles. To give but a few examples, detectable signals may include, but are not limited to, signals from radioisotopes, fluorophores, chromophores, electron dense particles, magnetic particles, spin labels, molecules that emit chemiluminescence, electrochemically active molecules, enzymes, cofactors, enzymes linked to nucleic acid probes and enzyme substrates.

In some embodiments, a detectable property of and/or around the particle is generated or changed as a result of nucleic acid capture and/or amplification. Those of ordinary skill in the art will appreciate that, in some embodiments, actual detection or development of a generated or changed detectable property may require or involve one or more additional steps (e.g., binding of a labeled moiety to an amplified nucleic acid, or to a particle containing an amplified nucleic acid, interaction with a reactant (e.g., electromagnetic radiation, an enzyme, a reagent, or a combination of these) that triggers a detectable event from the amplified nucleic acid or the particle or an entity with which one of these has interacted, etc). Such steps are well known in the art.

In some embodiments, a detectable property (aspect) is optical. Exemplary optical properties include, but are not limited to, fluorescent, ultraviolet, infrared, holographic, radiographic signals and any combination thereof. An optical property, in some embodiments, can be detected through absorption, emission, reflection, refraction, interference, diffraction, dispersion, scattering, or any combination thereof, etc.

According to the present invention, detection and/or quantification can comprise a step of counting the number of particles that contain a captured target nucleic acid. Such counting can determine the quantity of target nucleic acids in samples. In some embodiments, the quantity of a target nucleic acid is about the number of particles that contains a captured target nucleic acid. In some embodiments, the quantity is about half of the number of particles that contains a captured target nucleic acid (e.g., when each strand of an initially double-stranded target is separately captured, for example, on a different particle so that two particles in effect the same initially double stranded target).

In many embodiments, counting involves distinguishing particles with captured target nucleic acid molecules from those without captured target nucleic acid molecules by detecting an optical aspect or property that distinguishes particles with captured targets from those without captured targets. That is, the present invention provides methodologies that permit detection of those particles that contain amplified (e.g., bridged) target nucleic acid molecules by detection of generation of an optical property, or a change in an optical property, of and/or around the particle/amplified target complex as compared with particles that lack amplified target nucleic acids.

In certain embodiments, detection and/or quantification used in accordance with the present invention does not require (and/or does not involve) quantification of copies of a target nucleic acid and/or its amplified products on a particle. In certain embodiments, detection and/or quantification used herein does not require (and/or does not involve) capture of nucleic acids that have some common sequences but with different lengths. In certain embodiments, detection and/or quantification used herein does not require (and/or does not involve) determination of size distribution of different species (e.g., different lengths of nucleic acids) present in a sample.

### Optical Characterization

In some embodiments, optical characterization is used in accordance with the present invention. Illustrative optical detection methodologies include, but are not limited to, light scattering, multichannel fluorescence detection, UV and visible wavelength absorption, luminescence, differential reflectivity, and confocal laser scanning. Additional detection methods that can be used in certain applications include scintillation proximity assay (SPA) techniques, radiochemical detection, fluorescence polarization, fluorescence correlation spectroscopy (FCS), time-resolved energy transfer (TRET), fluorescence resonance energy transfer (FRET) and variations such as bioluminescence resonance energy transfer (BRET). Additional or alternative detection options include electrical resistance, resistivity, impedance, and voltage sensing.

Detection and/or quantification, in many embodiments, in accordance with the present invention may be carried out using any suitable detection device. Particles with captured and/or amplified nucleic acids may be detected by an optical means. Commercially available optical reading systems, for example, fluorescence detectors, can be used.

In some embodiments, after being contacted with sample and/or exposed to PCR conditions, for example, individual particles are analyzed by flow-through reading. A flow-through device suitable for use in the present invention can be a flow cytometer. Flow cytometry is a technique routinely used in diagnosis for counting and/or examining particles, typically in microscopic range, by suspending them in a stream of fluid and passing them by an electronic detection apparatus. In some embodiments, flow cytometry is used to measures properties such as light scattering and/or fluorescence on individual particles in a flowing stream, allowing a population or more than one subpopulation of particles within a sample to be identified, analyzed, and optionally characterized. In certain embodiments, laser scanning cytometry is used. Laser scanning cytometers are available, e.g., from CompuCyte (Cambridge, Mass.).

In some embodiments, individual particles are distributed onto a substrate for imaging. For example, particles can be spread on a substrate or distributed into discrete areas on a substrate. In certain embodiments, discrete areas on a substrate contain, on average, no more than a single particle per area. In some embodiments, imaging systems comprising an epifluorescence microscope equipped with a laser (e.g., a 488 nm argon laser) for excitation and appropriate emission filter(s) are used. The filters should allow discrimination between different populations of particles used in a particular assay.

In some embodiments, a step of optically characterizing involves detecting a generation or a change of an optical property of and/or around a particle Optical property of a particle can be altered directly or indirectly (e.g., requiring or involving one or more additional steps) for determination and/or quantification.

In some embodiments, detection may include contacting particles with an intercalation dye that has dramatic fluorescent enhancement upon binding to double-stranded DNA. Such dyes can be used, for example, to detect captured and/or amplified nucleic acids. Examples of suitable dyes include, but are not limited to, SYBR™ and Pico Green (from Molecular Probes, Inc. of Eugene, Oreg.), ethidium bromide, propidium iodide, chromomycin, acridine orange, Hoechst 33258, Toto-1, Yoyo-1, and DAPI (4′,6-diamidino-2-phenylindole hydrochloride). Additional discussion regarding the use of intercalation dyes is provided by Zhu et al., Anal. Chem. 66:1941-1948 (1994), which is incorporated by reference in its entirety.

In some embodiments, detection includes cleavage of a double-strand nucleic acid (e.g., a PCR product). For example, particles may be contacted with a restriction enzyme (e.g., endonuclease). In some embodiments, detection includes release from a double-strand of at least a single strand of a captured and/or amplified nucleic acid. In some embodiments, a single strand remains associated with the particle and can be detected, for example, by hybridization or other means.

In some embodiments, a probe containing a reporter dye (e.g., fluorescent or luminescent dyes), which hybridizes with a captured and/or amplified nucleic acid is used. Typically, a fluorophore is an aromatic or heteroaromatic compound and can be a pyrene, anthracene, naphthalene, acridine, stilbene, indole, benzindole, oxazole, thiazole, benzothiazole, canine, carbocyanine, salicylate, anthranilate, coumarin, fluorescein, rhodamine or other like compound. Exemplary fluorescent reporters include xanthene dyes, such as fluorescein or rhodamine dyes, including 6-carboxyfluorescein (FAM), 2′7′-dimethoxy-4′5′-dichloro-6-carboxyfluorescein (JOE), tetrachlorofluorescein (TET), 6-carboxyrhodamine (R6G), N,N,N; N′-tetramethyl-6-carboxyrhodamine (TAMRA), 6-carboxy-X-rhodamine (ROX). Exemplary fluorescent reporters also include the naphthylamine dyes that have an amino group in the alpha or beta position. For example, naphthylamino compounds include 1-dimethylaminonaphthyl-5-sulfonate, 1-anilino-8-naphthalene sulfonate and 2-p-toluidinyl-6-naphthalene sulfonate, 5-(2′-aminoethyl)aminonaphthalene-1-sulfonic acid (EDANS). Other fluorescent reporter dyes include coumarins, such as 3-phenyl-7-isocyanatocoumarin; acridines, such as 9-isothiocyanatoacridine and acridine orange; N-(p-(2-benzoxazolyl)phenyl) maleimide; cyanines, such as indodicarbocyanine 3 (Cy3), indodicarbocyanine 5 (Cy5), indodicarbocyanine 5.5 (Cy5.5), 3-(-carboxy-pentyl)-3′-ethyl-5,5′-dimethyloxacarbocyanine (CyA); 1H, 5H, 11H, 15H-Xantheno[2,3, 4-ij: 5,6, 7-i′j′]diquinolizin-18-ium, 9-[2 (or 4)-[[[6-[2,5-dioxo-1-pyrrolidinyl)oxy]-6-oxohexyl]amino]sulfonyl]-4 (or 2)-sulfophenyl]-2,3,6,7, 12,13,16,17-octahydro-inner salt (TR or Texas Red); BODIPYTM dyes; benzoxaazoles; stilbenes; pyrenes; and the like. Descriptions of fluorophores and their use, can be found in, among other places, R. Haugland, Handbook of Fluorescent Probes and Research Products, 9^{th} ed. (2002), Molecular Probes, Eugene, Oreg.; M. Schena, Microarray Analysis (2003), John Wiley & Sons, Hoboken, N.J.; Synthetic Medicinal Chemistry 2003/2004 Catalog, Berry and Associates, Ann Arbor, Mich.; G. Hermanson, Bioconjugate Techniques, Academic Press (1996); and Glen Research 2002 Catalog, Sterling, Va. Near-infrared dyes are expressly within the intended meaning of the terms fluorophore and fluorescent reporter group.

In certain embodiments, a probe is a molecular beacon (MB) probe. In general, MB probes are oligonucleotides with stem-loop structures that contain a fluorescent dye at the 5′ end and a quenching agent (Dabcyl) at the 3′ end. The degree of quenching via fluorescence energy resonance transfer is inversely proportional to the 6th power of the distance between the Dabcyl group and the fluorescent dye. After heating and cooling, MB probes reform a stem-loop structure, which quenches the fluorescent signal from the dye. According to the present invention, if a captured and/or amplified nucleic acid whose sequence is complementary to the loop sequence is present during the heating/cooling cycle, hybridization of the MB to one strand of the captured and/or amplified nucleic acid will increase the distance between the Dabcyl and the dye, resulting in increased fluorescence. A wide variety of reactive fluorescent reporter dyes are known in the literature and can be used, in some embodiments of the present invention, so long as they are quenched by the corresponding quencher dye of the invention. For further details, see WO/2005/049849, “fluorescence quenching azo dyes, their methods of preparation and use”, the contents of which is incorporated by references.

### Encoding

A variety of different particles as described above can be of use in accordance with the invention. In some embodiments, a population of particles used in accordance with the present invention has more than one subgroup of particles. A subgroup of particles can share a signature on individual particles in the subgroup to be differentiated from another subgroup of particles. Such encoding enables multiplexed analysis of more than one type of targeted nucleic acids.

Detectable signatures, and particularly optically detectable signatures, can be used in accordance with the present invention for encoding different subpopulations of particles. In some embodiments, each such subpopulation of particles carries one particular primer pair and that primer pair is different from the primer pair carried by other subpopulations. In some embodiments, the primer pair on each subpopulation is designed specifically for capture and/or amplification of one particular target nucleic acid in a sample (e.g., by bridge PCR). Such encoding, in general, enables multiplexed analysis, that is, detecting and/or quantifying more than one type of target nucleic acids in a sample. Encoding and/or decoding can be performed separately from (e.g., prior to and/after) or simultaneously with capture and/or amplification of target nucleic acids. Similarly, detection of encoded signatures can be performed separated from (e.g., prior to and/after) or simultaneously with the detection of captured and/or amplified nucleic acids.

It is not intended that the present invention be limited to a particular coding scheme. A signature for encoding can be a visually detectable feature such as, for example, color, apparent size, or visibility (i.e. simply whether or not the particle is “visible”, or optically detectable, under particular conditions). Such visibility, as will be understood by those skilled in the art, can include, for example, presence or amount of electromagnetic radiation at one or more particular frequencies, presence or identity of a particular holographic signature, presence or amount of radioactivity, etc. In various embodiments of the present invention, an optical signature of a particle is used for encoding. Detailed description of optically interrogatable encoding can be found, for example, in U.S. Pat. No. 6,023,540 and U.S. Pat. No. 6,327,410, the contents of which are incorporated herein by reference.

In some embodiments, an optical signature for encoding is or comprises a feature of an absorption, emission, reflection, refraction, interference, diffraction, dispersion, scattering, or any combination thereof. In some embodiments, an optical signature is or comprises a change in a feature of absorption, emission, reflection, refraction, interference, diffraction, dispersion, scattering, or any combination thereof under suitable conditions.

In some embodiments, an optical signature is intrinsic to utilized particles in accordance with the present invention. In some embodiments, an optical signature is introduced to particles. Such introduction can be done before, with or after capture and/or amplification of target nucleic acids.

For example, a dye (e.g., a fluorescent dye) can be introduced to particles. In some embodiments, dyes can be covalently bonded to particles. In some embodiments, dyes can be physically associated with particles. Typically, particles can be placed in a dye solution comprising a ratio of two or more dyes. Particles may swell in the solution and be doped with dyes.

In an exemplary two dye system, Texas Red Cadaverine (TRC) can be used, which is excited at λ_{ab}=580 mm and emits at λₑₘ=630 mm, in combination with indodicarbocyanine (DiIC): 610/670 (λ_{ab}/λₑₘ). Generally, dyes may be selected to be compatible with other functionalities and to be spectrally compatible. Examples of other dyes that can be used are Oxazin (662/705), IR-144 (745/825), IR-140 (776/882), IR-125 (786/800) from Exiton, and Bodipy 665/676 from Molecular Probes, and Naphthofluorescein (605/675) also from Molecular Probes. Lanthanides may also be used. Fluorescent dyes emitting in other than the near infrared may also be used. Chromophore dyes are still another alternative that produce an optically detectable signature, as are more exotic formulations using Raman scattering-based dyes or polarizing dyes, for example.

The ability of a particular dye pair for encoding depends on the resolution of the ratiometric measurement. Conservatively, any dye pair should provide the ability to discriminate at least twenty different ratios. Furthermore, combining more than two dyes provides additional diversity in encoding combinations.

To give but another example, in some embodiments a particle carries a moiety that is not itself optically active for encoding, but upon interaction with and/or modification by or to another particular moiety (e.g., a decoder), becomes optically active. One particular embodiment of such an approach involves use of a fluorescence-labeled or otherwise detectable decoder moiety, which can be or comprise a decoder oligonucleotide. The sequence of such a decoder oligonucleotide can be complementary to that of an oligonucleotide on a particle. Particles containing such oligonucleotides can be contacted with detectable decoder oligonucleotides before, during and/or after interaction with targets. Further details can be found, for example, in K. Gunderson et al. “Decoding Randomly Ordered DNA Arrays” Genome Research, 14:870-877, 2004, which is incorporated by reference herein.

In some embodiments, particles are encoded with a holographic code. When excited with a light (e.g., a laser), such a particle emits a specific holographic code, image or pattern that can, for example, distinguish it from particles comprising different holographic codes.

### Applications

The present invention has many applications, including, but not limited to, diagnosis and monitoring in medicine and any non-medical applications, where the presence and/or the amount of a target can be determined. In some embodiments, the presence or the amount of a target nucleic acid is determined using the present invention.

Those of ordinary skill reading the present disclosure, will appreciate its broad applicability. In some embodiments, provided methods herein are used to detect and/or quantify target nucleic acids, for example, to profile a specific tissue or a specific condition. In some embodiments, provided methods herein are used to detect and/or quantify target nucleic acids to detect biomarkers for specific tissue or condition. In certain embodiments, provided methods herein are used to detect and/or quantify target nucleic acids to profile a neoplastic and/or cancer cell.

For example, a wide variety of infectious diseases can be detected and/or determined by the process of the present invention, for example, those caused by bacterial, viral, parasite, and fungal infectious agents. The resistance of various infectious agents to drugs can also be determined using the present invention.

Representative bacterial infectious agents which can be detected and/or determined by the present invention include, but are not limited to, Escherichia coli, Salmonella, Shigella, Klebsiella, Pseudomonas, Listeria monocytogenes, Mycobacterium tuberculosis, Mycobacterium aviumintracellulare, Yersinia, Francisella, Pasteurella, Brucella, Clostridia, Bordetella pertussis, Bacteroides, Staphylococcus aureus, Streptococcus pneumonia , B-Hemolytic strep., Corynebacteria, Legionella, Mycoplasma, Ureaplasma, Chlamydia, Neisseria gonorrhea, Neisseria meningitides, Hemophilus influenza, Enterococcus faecalis, Proteus vulgaris, Proteus mirabilis, Helicobacter pylori, Treponema palladium, Borrelia burgdorferi, Borrelia recurrentis, Rickettsial pathogens, Nocardia, and Acitnomycetes.

Representative fungal infectious agents which can be detected and/or determined by the present invention include, but are not limited to, Cryptococcus neoformans, Blastomyces dermatitidis, Histoplasma capsulatum, Coccidioides immitis, Paracoccidioides brasiliensis, Candida albicans, Aspergillus fumigautus, Phycomycetes (Rhizopus), Sporothrix schenckii, Chromomycosis, and Maduromycosis.

Representative viral infectious agents which can be detected and/or determined by the present invention include, but are not limited to, human immunodeficiency virus, human T-cell lymphocytotrophic virus, hepatitis viruses (e.g., Hepatitis B Virus and Hepatitis C Virus), Epstein-Barr Virus, cytomegalovirus, influenza viruses, human papillomaviruses, orthomyxo viruses, paramyxo viruses, adenoviruses, corona viruses, rhabdo viruses, polio viruses, toga viruses, bunya viruses, arena viruses, rubella viruses, and reo viruses.

Representative parasitic agents which can be detected and/or determined by the present invention include, but are not limited to, Plasmodium falciparum, Plasmodium malaria, Plasmodium vivax, Plasmodium ovale, Onchoverva volvulus, Leishmania, Trypanosoma spp., Schistosoma spp., Entamoeba histolytica, Cryptosporidum, Giardia spp., Trichimonas spp., Balatidium coli, Wuchereria bancrofti, Toxoplasma spp., Enterobius vermicularis, Ascaris lumbricoides, Trichuris trichiura, Dracunculus medinesis, trematodes, Diphyllobothrium latum, Taenia spp., Pneumocystis carinii, and Necator americanis.

The present invention can also be useful for detection and/or determination of drug resistance by infectious agents. For example, vancomycin-resistant Enterococcus faecium, methicillin-resistant Staphylococcus aureus, penicillin-resistant Streptococcus pneumoniae, multi-drug resistant Mycobacterium tuberculosis, and AZT-resistant human immunodeficiency virus can be identified with the present invention.

Genetic diseases can also be detected and/or determined by the process of the present invention. This can be carried out by prenatal or post-natal screening for chromosomal and genetic aberrations or for genetic diseases. Examples of detectable genetic diseases include, but are not limited to: 21 hydroxylase deficiency, cystic fibrosis, Fragile X Syndrome, Turner Syndrome, Duchenne Muscular Dystrophy, Down Syndrome or other trisomies, heart disease, single gene diseases, HLA typing, phenylketonuria, sickle cell anemia, Tay-Sachs Disease, thalassemia, Klinefelter Syndrome, Huntington Disease, autoimmune diseases, lipidosis, obesity defects, hemophilia, inborn errors of metabolism, and diabetes.

Cancers which can be detected and/or determined by the process of the present invention generally involve oncogenes, tumor suppressor genes, or genes involved in DNA amplification, replication, recombination, or repair. Examples of these include, but are not limited to: BRCA1 gene, p53 gene, APC gene, Her2/Neu amplification, Bcr/Ab1, K-ras gene, and human papillomavirus Types 16 and 18. Various aspects of the present invention can be used to identify amplifications, large deletions as well as point mutations and small deletions/insertions of the above genes in the following common human cancers: leukemia, colon cancer, breast cancer, lung cancer, prostate cancer, brain tumors, central nervous system tumors, bladder tumors, melanomas, liver cancer, osteosarcoma and other bone cancers, testicular and ovarian carcinomas, head and neck tumors, and cervical neoplasms.

In the area of environmental monitoring, the present invention can be used, for example, for detection, identification, and monitoring of pathogenic and indigenous microorganisms in natural and engineered ecosystems and microcosms such as in municipal waste water purification systems and water reservoirs or in polluted areas undergoing bioremediation. It is also possible to detect plasmids containing genes that can metabolize xenobiotics, to monitor specific target microorganisms in population dynamic studies, or either to detect, identify, or monitor genetically modified microorganisms in the environment and in industrial plants.

The present invention can also be used in a variety of forensic areas, including, for example, for human identification for military personnel and criminal investigation, paternity testing and family relation analysis, HLA compatibility typing, and screening blood, sperm, or transplantation organs for contamination.

In the food and feed industry, the present invention has a wide variety of applications. For example, it can be used for identification and characterization of production organisms such as yeast for production of beer, wine, cheese, yoghurt, bread, etc. Another area of use is with regard to quality control and certification of products and processes (e.g., livestock, pasteurization, and meat processing) for contaminants. Other uses include the characterization of plants, bulbs, and seeds for breeding purposes, identification of the presence of plant-specific pathogens, and detection and identification of veterinary infections.

### Kits

Also provided are kits for carrying out the methods described herein. In some embodiments, a kit may comprise a sufficient quantity of particles with primer pairs in a population to perform an amplification reaction on at least a target nucleic acid from a sample. A population of particle can have one or more copies of one type of primer pairs on each particle. In some embodiments, a kit comprises more than one subpopulation of particles, each having one or more copies of one type of primer pairs.

Primer pairs on different particles can be different. In some embodiments, a kit comprises more than one type of primer pairs. In some embodiments, a kit comprises from one to twenty types of primer pairs, from one to ten types of primer pairs, from one to eight types of pairs, from one to five types of primer pairs, from one to three types of primer pairs, or from one to two types of primer pairs for multiplexing.

In some embodiments, a kit comprises one or more reagents for optical characterization. For example, a fluorescent or other optically labeled probes that comprise at least a complementary sequence to an amplified nucleic acid on a particle used in accordance with the methods herewith.

In some embodiments, a kit comprises a sufficient quantity of reverse transcriptase, a DNA polymerase, suitable nucleoside triphosphates (including any of those described above), a DNA ligase, and/or reaction buffer, or any combination thereof, for the amplification processes described above. For example, with target RNAs, a reverse transcription step can be performed prior to amplification. In certain embodiments, a target RNA is amplified by a reverse transcription step followed by a DNA amplification step using different enzymes. In certain embodiments, a target RNA is reversed transcribed and the resulting DNA is amplified using an enzyme, such as the Thermus thermophilus (Tth) polymerase that possesses both reverse transcriptase and DNA polymerase functions.

In some embodiments, a kit may contain one or more restriction enzymes (e.g., endonucleases) for cleaving one or more bridge-amplified double stranded DNAs.

A kit may include instructions pertinent for the particular embodiment of the kit, such instructions describing the primer pairs and amplification conditions for operation of the method. In some embodiments, the kit further comprises instructions for analysis, interpretation and dissemination of data acquired by the kit. In some embodiments, instructions for the operation, analysis, interpretation and dissemination of the data of the kit are provided on computer readable media. A kit may also comprise amplification reaction containers such as microcentrifuge tubes, microtiter plates, and the like. A kit may also comprise reagents or other materials for preparing samples and/or performing methods, including, for example, detergents, solvents, or ion exchange resins which may be linked to magnetic beads.

### EXEMPLIFICATION

### Example 1

### Quantifying Transcripts

According to the present invention, provided single-molecule bridge PCR methodologies are particularly useful in quantifying transcript (e.g., primary transcripts, mRNA, etc.) nucleic acids.

miRNA samples can be obtained from any tissue according to standard techniques known in the art. For instance, samples can be obtained from blood. Experimental details can be found, for example, in US 2009018139 which is incorporated by reference herein, and can be used in accordance with the present invention with or without modification and optimization.

In some embodiments, a crude sample is analyzed according to the prevent invention, and it requires and/or involves no further purification of targets (e.g., miRNA in this Example). However, in some embodiments a further isolation step may be performed. In order to perform this purification, a sample can be further purified according to standard techniques known in the art.

### Other Embodiments and Equivalents

While the present disclosures have been described in conjunction with various embodiments and examples, it is not intended that they be limited to such embodiments or examples. On the contrary, the disclosures encompass various alternatives, modifications, and equivalents, as will be appreciated by those of skill in the art. Accordingly, the descriptions, methods and diagrams of should not be read as limited to the described order of elements unless stated to that effect.

Although this disclosure has described and illustrated certain embodiments, it is to be understood that the disclosure is not restricted to those particular embodiments. Rather, the disclosure includes all embodiments that are functional and/or equivalents of the specific embodiments and features that have been described and illustrated.

We claim:

1. A method comprising steps of:
a) providing a population of particles wherein the population of the particles each carries one or more copies of a single pair of primers, which pair comprises a capture/first primer and a second primer for amplification of a target nucleic acid;
b) contacting the population with a sample comprising a quantity of the target nucleic acid, under conditions that permit the target nucleic acid in the sample to hybridize with the capture/first primer, thereby being captured to the particles, the step of contacting further being performed under conditions so that, on average, not more than one copy of the target nucleic acid from the sample hybridizes to any individual particle;
c) performing at least bridge PCR on the particles so that one or more optical aspects of those particles that hybridize and amplify the target nucleic acid are altered, and the particles that contain at least one copy of an amplified nucleic acid of the target nucleic acid are optically distinguishable from those that do not;
d) optically characterizing the population, so as to determine the number of particles that contain the at least one amplified nucleic acid of the target nucleic acid, which number reflects the quantity of the target nucleic acid in the sample.

2. The method of claim 1, wherein the one or more optical aspects are or comprise fluorescence.

3. The method of claim 1 or 2, wherein the target nucleic acid is selected from the group consisting of DNA, RNA, miRNA, cDNA and any combination thereof.

4. The method of claim 3, wherein the target nucleic acid is miRNA.

5. The method of claim 4, further comprising a step of reverse transcription prior to the step b).

6. The method of any one of claims 1-5, wherein the step c) comprises contacting with an intercalating dye.

7. The method of any one of claims 1-5, wherein the step of c) comprises contacting with a restriction endonuclease to cleave a strand of a double-stranded nucleic acid.

8. The method of claim 7, further comprising hybridizing with a complementary sequence to the other strand of the double-stranded nucleic acid.

9. The method of any one of claims 1-8, wherein the step d) is performed by flow cytometry.

10. The method of any one of claims 1-8, wherein the step d) is performed by imaging.

11. The method of any one of claims 1-10, wherein the sample is selected from the group consisting of blood, plasma, serum, saliva, tissue and any combination thereof.

12. The method of any one of claims 1-11, wherein the sample is from a cancer patient.

13. The method of any one of claims 1-12, wherein the target nucleic acid is or comprises at least a portion of a gene related to a genetic disease or a genetic polymorphism.

14. The method of any one of claims 1-13, wherein the target nucleic acid is or comprises at least a portion of an oncogene or a tumor suppressor gene.

15. The method of any one of claims 1-14, wherein the target nucleic acid is or comprises at least a portion of a virus genome.

16. The method of any one of claims 1-15, wherein the particles are encoded.

17. The method of claim 16, further comprising a step of decoding the encoded particles.

18. A method comprising steps of:
a) providing a population of particles that comprises one or more sub-populations, the sub-populations differing from one another in that:
i) each sub-population has an optical signature distinguishable from one another; and
ii) each sub-population carries one or more copies of a single pair of primers, which pair comprises a capture/first primer and a second primer for amplification of a particular target nucleic acid;
b) contacting the population with a sample comprising quantities of one or more target nucleic acids, under conditions that permit the one or more target nucleic acids in the sample to hybridize with their cognate capture/first primers, thereby being captured respectively to the particles in the one or more subpopulations, the step of contacting further being performed under conditions so that, on average, not more than one copy of the one or more target nucleic acids from the sample hybridizes to any individual particle;
c) performing bridge PCR on the particles so that one or more optical aspects of those particles that hybridize and amplify the target nucleic acids are altered, and the particles that contain at least one copy of a cognate amplified nucleic acid of the target nucleic acids are optically distinguishable from those that do not;
d) optically characterizing each sub-population of the population, so as to determine the number of particles that contain the at least one amplified nucleic acid of the one or more target nucleic acids, which number reflects the quantity of the one or more target nucleic acids in the sample.

19. The method of claim 18, wherein the one or more optical aspects of each sub-population are or comprise fluorescence.

20. The method of claim 18 or 19, wherein the one or more target nucleic acids are independently selected from the group consisting of DNA, RNA, miRNA, cDNA and any combination thereof.

21. The method of claim 20, wherein the one or more target nucleic acids are or comprise miRNA.

22. The method of claim 21, further comprising a step of reverse transcription prior to the step b).

23. The method of any one of claims 18-22, wherein the step c) comprises contacting with an intercalating dye.

24. The method of any one of claims 18-22, wherein the step of c) comprises contacting with a restriction endonuclease to cleave a strand of a double-stranded nucleic acid.

25. The method of claim 24, further comprising hybridizing with a complementary sequence to the other strand of the double-stranded nucleic acid.

26. The method of any one of claims 18-25, wherein the step d) is performed by flow cytometry.

27. The method of any one of claims 18-25, wherein the step d) is performed by imaging.

28. The method of any one of claims 18-27, wherein the sample is selected from the group consisting of blood, plasma, serum, saliva, tissue and any combination thereof.

29. The method of any one of claims 18-28, wherein the sample is from a cancer patient.

30. The method of any one of claims 18-29, wherein the one or more target nucleic acids are or comprise at least a portion of a gene related to a genetic disease or a genetic polymorphism.

31. The method of any one of claims 18-30, wherein the one or more target nucleic acids are or comprise at least a portion of an oncogene or a tumor suppressor gene.

32. The method of any one of claims 18-31, wherein the one or more target nucleic acids are or comprise at least a portion of a virus genome.

33. A kit comprising:
a) a population of particles wherein the particles each carries one or more copies of a single pair of primers, which pair comprises a capture/first primer and a second primer for amplification of a target nucleic acid; and
b) a polymerase that amplifies the target nucleic acid.

34. A kit comprising:
a) a population of particles comprising one or more sub-populations, wherein the sub-populations differing from one another in that:
i) each sub-population has an optical signature distinguishable from one another; and
ii) each sub-population carriers one or more copies of a single pair of primers, which pair comprises a capture/first primer and a second primer for amplification of a particular one of one or more target nucleic acids; and
b) one or more polymerases that amplify the one or more target nucleic acids.

35. The kit of claim 33 or 34, further comprising one or more restriction enzymes.
